(19) United States
(12) Patent Application Publication
(10) Pub. No.: US 2021/0228308 A1
(43) Pub. Date: Jul. 29, 2021

(54) REPRESENTATION APPARATUS

(71) Applicant: Siemens Healthcare GmbH, Erlangen (DE)

(72) Inventors: Martin Berger, Erlangen (DE); Christian Kaethner, Forchheim (DE); Tobias Lenich, Nürnberg (DE); Alois Regensburger, Poxdorf (DE)

(21) Appl. No.: 17/159,391

(22) Filed: Jan. 27, 2021

(30) Foreign Application Priority Data
Jan. 29, 2020 (DE) 10 2020 201 070.6

(51) Int. Cl. A61B 90/00; G06T 7/00

(52) U.S. Cl. CPC A61B 90/361; A61B 2090/366; A61B 2090/365; G06T 7/0012

(57) ABSTRACT

A representation apparatus configured to represent an augmented and/or a virtual reality, wherein the representation apparatus comprises a camera unit that is configured for mapping, at least in segments, an examination subject arranged in a direction of view of the representation apparatus. The camera unit is further configured for mapping a light pattern projected onto the examination subject by a projection unit. The light pattern includes a defined projection geometry relative to the projection unit. The representation apparatus is configured to adjust the representation of the augmented and/or virtual reality using the mapped light pattern and the defined projection geometry.

### CROSS REFERENCE TO RELATED APPLICATIONS

This application claims the benefit of DE 102020201070.6 filed on Jan. 29, 2020, which is hereby incorporated by reference in its entirety.

### FIELD

Embodiments relate to a representation apparatus for medical information.

### BACKGROUND

For the realistic representation of medical information, for example of medical image data from an examination subject, representations of an augmented and/or virtual reality (abbreviated to AR and VR respectively) are increasingly used. This often involves real objects, for example medical objects and/or an examination subject, being superimposed with virtual data, for example with medical image data and/or virtual objects and represented in a display. For a realistic representation with a high level of immersion, a precise registration of the virtual data and the real objects is required.

For this purpose, the real object that is to be embedded in the representation of the augmented and/or virtual reality is located using a physical marker structure arranged on the object. Alternatively, the real object may be located using a pre-determined shape relative to an apparatus for representing the augmented and/or virtual reality. The disadvantage with this, however, is often the prior knowledge of the shape of the real object that is necessary, for example with different examination subjects and/or examination regions, and/or fitting the marker structure to the real object.

Moreover, the for example current, position and/or alignment of the apparatus for representing the augmented and/or virtual reality relative to the real object may be determined by a marker structure that is arranged on the apparatus and/or by integrated sensors. The monitoring of the marker structure that is arranged on the apparatus disadvantageously often requires a latency-free and stable data transfer between a relevant monitoring system and the apparatus. Furthermore, an accuracy of the determination of the position and/or alignment of the apparatus by the integrated sensors is often insufficient and prone to failure.

### BRIEF SUMMARY AND DESCRIPTION

The scope of the present disclosure is defined solely by the appended claims and is not affected to any degree by the statements within this summary. The present embodiments may obviate one or more of the drawbacks or limitations in the related art.

Embodiments address the problem of facilitating a precise and reliable registration between a representation of an augmented and/or virtual reality and an examination subject.

In an embodiment, a representation apparatus for displaying an augmented and/or virtual reality is provided. The representation apparatus includes a camera unit that is configured for at least partially mapping an examination subject that is arranged in a viewing direction of the representation apparatus. The examination subject may be, for example, a human and/or animal patient and/or an examination phantom, for example an X-ray phantom and/or an MRT phantom and/or a workpiece. Furthermore, the camera unit is configured for mapping using a light pattern projected onto the examination subject by a projection unit. The light pattern includes a defined projection geometry relative to the projection unit. Furthermore, the representation apparatus is configured to adjust the representation of the augmented and/or virtual reality using the light pattern that has been mapped and the projection geometry that has been defined.

The representation apparatus may additionally include a portable display unit, for example a display unit that may be carried by a user and that is configured to display the augmented and/or virtual reality (abbreviated to AR and VR respectively). The representation apparatus, for example the display unit, may be configured to be at least partly transparent and/or possible to see through. The display unit may be configured such that the display unit may be carried by a user at least partly within the user's field of view. The display unit may be configured as a pair of glasses, for example as data glasses, and/or as a helmet, for example as a data helmet, and/or as a screen.

The representation apparatus, for example the display unit, may be configured to be superimposed on real objects, for example medical objects and/or the examination subject, using virtual data, for example measured and/or simulated and/or processed medical image data and/or virtual objects, and represent them in a display.

The camera unit may include at least one camera, for example a 2D camera and/or an omni-directional camera and/or a 3D camera, for example a stereo camera and/or depth camera and/or a time-of-flight camera (TOF camera), that is configured for mapping, at least in segments, the examination subject, that is for example arranged in a direction of view of the user, in the direction of view of the representation apparatus. The camera unit may map at least one segment of a surface of the examination subject and provide a corresponding signal, for example including 2D- and/or 3D-image data, to a processing unit.

The projection unit may include at least one projector, for example a light projector and/or a laser projector, that is configured for projecting the light pattern onto the examination subject, for example onto a surface of the examination subject. The projection unit, for example the at least one projector, may be arranged apart from the representation apparatus, for example fixedly and/or pivotably and/or rotatably, and/or on the representation apparatus. Furthermore, the projection unit may be affixed to a medical device, for example to a medical imaging device and/or to a patient-positioning apparatus. The representation apparatus may be configured to represent medical image data as augmented and/or virtual reality, it being possible for the medical image data to be mapped using the medical imaging device.

Furthermore, the light pattern may be configured such that it allows mapping of a surface shape of the at least one segment of the examination subject by the camera unit. When projecting the light pattern onto the examination subject by the projection unit, the three-dimensional surface shape of the examination subject may be mapped by distorting the light pattern from the camera unit in the representation apparatus. The light pattern may be created using the projection unit in a light wavelength region that is not visible to a human user.

Furthermore, the light pattern includes a defined projection geometry in relation to the projection unit. The projection geometry may include a defined dot pattern and/or a defined line pattern and/or structured light and/or a speckle pattern and/or a chessboard pattern and/or a randomized pattern and/or a defined arrangement of geometrical objects, that is specified for the projection of the light pattern onto the examination subject. Furthermore, the defined projection geometry may have a fan angle and/or a clearance with respect to a distance between the projection unit and the examination subject.

Furthermore, the projection unit may include a sensor unit to map a positioning of the examination subject relative to the projection unit, for example a camera and/or an ultrasound sensor and/or an electromagnetic sensor and/or an optical sensor and/or a laser scanner. The positioning of the examination subject may include information on the spatial position and/or on the spatial alignment of the examination subject. As a result, a coordinate system of the projection unit may be registered with a coordinate system of the examination subject. For example, the defined projection geometry may be registered with the coordinate system of the examination subject.

The defined projection geometry of the light pattern to the projection unit describes the geometry of the rays of light that are emitted to project the light pattern by the projection unit, for example by the at least one projector, onto the examination subject. The defined projection geometry may remain unchanged over time during the representation of the augmented and/or virtual reality of the representation apparatus. As a result, repeating the provision of the defined projection geometry to the representation apparatus may be avoided.

The light pattern on the surface of the examination subject may be mapped at least in segments by the camera unit. A coordinate system in the representation apparatus may be registered with the coordinate system of the examination subject, using the mapped light pattern and the defined projection geometry, that may be provided for example by the projection unit. As result of the projection unit, for example the coordinate system of the projection unit, being registered with the coordinate system of the examination subject, the coordinate system of the representation apparatus may be reliably registered with the coordinate system of the examination subject, even in the event of a change in the positioning of the examination subject, using the mapped light pattern and the defined projection geometry. For this purpose, the sensor unit in the projection unit may map the change in the positioning of the examination subject and provide the representation apparatus with the defined projection geometry that has been adjusted accordingly.

The proposed representation apparatus provides a precise and reliable registration between the examination subject and the representation apparatus, for example a precise adjustment of the representation of the augmented and/or virtual reality on the examination subject. It is possible to avoid applying physical marker objects to the examination subject and/or the representation apparatus.

Moreover, the light pattern projected by the projection unit may be configured such that it may be incorporated into the representation of the augmented and/or virtual reality, for example visibly.

Alternatively, or additionally, the registration between the coordinate system in the projection unit and the coordinate system for the examination subject may be facilitated by the projection unit's being arranged in a defined positioning on a medical device, the medical device being configured to map the positioning of the examination subject relative to the medical device.

Furthermore, the projected light pattern may be adaptable, for example over time. As a result, for example, an improved, for example chronological, synchronization between the projection unit and the representation apparatus may be facilitated.

Furthermore, the representation apparatus may include a positioning sensor that is configured to map a spatial positioning of the representation apparatus. The spatial positioning of the representation apparatus may include information on the spatial position and/or alignment, for example on the direction of view, of the representation apparatus. The positioning sensor may include, for example, a gyroscopic sensor and/or an electromagnetic sensor and/or an optical sensor.

As far as the projected light pattern is at least partly detectable by the camera unit, the representation of the augmented and/or virtual reality may be adjusted to the examination subject, for example to the current positioning of the examination subject and/or to the surface of the examination subject.

In a further advantageous embodiment of the proposed representation apparatus, the representation apparatus may be configured to reconstruct a 3D model of a surface of the examination subject using the light pattern and the defined projection geometry. The representation of the augmented and/or virtual reality may be additionally adjustable using the 3D model.

The 3D model of the surface of the examination subject, may be configured, for example, as a mesh model. The surface of the examination subject, for example the surface shape of the examination subject that has been mapped at least in segments, may be reconstructable using a comparison between the defined projection geometry and the mapped light pattern.

As a result of the coordinate system of the representation apparatus being registered with the coordinate system of the examination subject, the representation of the augmented and/or virtual reality may be adjusted in a particularly precise manner to the surface of the examination subject using the reconstructed 3D model of the surface of the examination subject. The representation of the augmented and/or virtual reality may include for example medical image data, the representation of the image data being adjustable to the surface of the examination subject by the reconstructed 3D model. With 3D image data for example, a precise representation of slice images and/or segmented anatomical structures may be facilitated by the reconstructed 3D model of the surface of the examination subject.

In an embodiment of the representation apparatus, the representation apparatus may be further configured to graphically represent, as the augmented and/or virtual reality, medical image data from the examination subject, that have been acquired using a medical imaging device and/or are provided by the device.

The medical image data may be for example two-dimensional and/or three-dimensional. The medical imaging device may be configured, for example, as a medical X-ray device and/or magnetic resonance tomography unit (MRT) and/or as a computer tomography unit (CT) and/or an ultrasound device and/or a positron emission tomography unit (PET).

Furthermore, the provision of the medical image data may include, for example, storage on a computer-readable storage medium and/or transfer to the representation apparatus and/or to a processing unit. For example, the medical image data may be graphically representable precisely as the augmented and/or virtual reality relative to the examination subject, for example to the coordinate system of the examination subject.

The graphic representation of the augmented and/or virtual reality may be based on pre-operative and/or intraoperative medical image data. Furthermore, the medical image data may be mapped using the same or using various medical imaging devices. For example, the pre-operative medical image data may have identical or different dimensions from the intraoperative medical image data. The medical image data may be processed, for example registered and/or segmented, for the graphic representation of the augmented and/or virtual reality, and/or at least partly simulated. In addition, virtual objects, for example a virtual medical object, for example a guide wire and/or a catheter, in the graphic representation of the augmented and/or virtual reality may be superimposed with the medical image data. For example, the virtual object is displayable in the graphic representation of the augmented and/or virtual reality, according to a spatial positioning of a medical object that is arranged in an examination region of the examination subject.

In an embodiment of the proposed representation apparatus, the camera unit may be configured to map the light pattern indirectly via a reflection unit. The reflection unit may have a known spatial positioning relative to the projection unit.

The reflection unit may be configured, for example, as a reflector and/or mirror and/or semi-transparent disc and/or projection screen and/or screen. The reflection unit is configured to facilitate indirect mapping of at least one segment of the light pattern on the surface of the examination subject. The reflection unit may be arranged apart from the examination subject such that the reflection unit is substantially arranged outside a direct direction of view of the representation apparatus towards the examination subject.

As far as a visual obstruction occurs in the direction of view of the representation apparatus. The visual obstruction prevents an at least partial direct mapping of the projected light pattern by the camera unit, the representation of the augmented and/or virtual reality may be adjustable to the examination subject, using the light pattern that has been mapped indirectly via the reflection unit. This is facilitated for example by the reflection unit including a spatial positioning that is known, for example defined, relative to the projection unit. The spatial positioning of the reflection unit includes information about the spatial positioning and/or alignment of the reflection unit, for example relative to the projection unit. As a result, the reflection geometry between the projected light pattern and the reflection unit is defined.

A field of view of the camera unit may be configured such that the indirect mapping of the light pattern is possible via the reflection unit even in a direction of view of the representation apparatus that is substantially averted from the reflection unit. Furthermore, the representation apparatus may be configured to adapt the adjustment of the representation of the augmented and/or virtual reality, both using the directly mapped light pattern and the indirectly mapped light pattern. As a result, accuracy in the adjustment of the representation of the augmented and/or virtual reality may be improved.

Alternatively, or additionally, the camera unit may be configured to map the positioning of the reflection unit. The positioning of the reflection unit may be mapped by the camera unit, using a defined shape and size of the reflection unit and/or using at least one marker object, that is arranged on the reflection unit and/or is incorporated therein. For example, in the case of a substantially rectangular and/or circular defined shape and a known spatial extent of the reflection unit, for example using a perspective distortion and/or tilting, the positioning of the reflection unit is determinable using an at least two-dimensional mapping by the camera unit.

A second aspect relates to a projection unit for projecting a light pattern onto a surface of an examination subject. The projection unit includes a projector that is configured for projecting the light pattern onto the surface of the examination subject. Furthermore, the light pattern includes a defined projection geometry relative to the projection unit. Moreover, the projection unit is configured for providing the defined projection geometry to a proposed representation apparatus.

The advantages of the proposed projection apparatus correspond to the advantages of the proposed representation apparatus. Features, advantages, or alternative embodiments may also be equally well applied to the other claimed subject matter and vice versa.

The projection unit may include at least one projector, for example a light projector and/or a laser projector, that is configured for projecting the light pattern onto the examination subject, for example onto a surface of the examination subject. The projection unit, for example the at least one projector, may be arranged apart from the proposed representation apparatus, for example fixedly and/or pivotably and/or rotatably, and/or on a proposed representation apparatus. Furthermore, the projection unit may be affixed to a medical device, for example to a medical imaging device.

Furthermore, the light pattern may be configured such that it facilitates mapping of a surface shape of the at least one segment of the examination subject using the camera unit in a proposed representation apparatus. Furthermore, the light pattern includes a defined projection geometry relative to the projection unit. The projection geometry may include a defined dot pattern and/or a defined line pattern and/or a randomized pattern and/or a defined arrangement of geometrical objects, that is specified for the projection of the light pattern onto the examination subject. Furthermore, the defined projection geometry may have a fan angle and/or a clearance with respect to a distance between the projection unit and the examination subject.

Furthermore, the projection unit may include a sensor unit to map a positioning of the examination subject relative to the projection unit, for example a camera and/or an ultrasound sensor and/or an electromagnetic sensor and/or an optical sensor and/or a laser scanner. The positioning of the examination subject may include information on the spatial position and/or on the spatial alignment of the examination subject. As a result, a coordinate system of the projection unit may be registered with a coordinate system of the examination subject. For example, the defined projection geometry may be registered with the coordinate system of the examination subject.

Furthermore, the provision of the defined medical projection geometry image data to a proposed representation apparatus may include, for example, storage on a computer-readable storage medium and/or a transfer to the proposed representation apparatus and/or to a processing unit.

As a result, a marker-free mapping of the examination subject, for example of a positioning of the examination subject and/or of a surface shape of the examination subject, may be facilitated by a mapping of the light pattern.

In an embodiment of the proposed projection apparatus, the light pattern may have a first and a second part. The projector may be configured for projecting the first part of the light pattern onto the surface of the examination subject. Furthermore, the projection unit may have at least one further projector, that is configured for projecting the second part of the light pattern onto the surface of the examination subject. The second part of the light pattern may include a different defined projection geometry than does the first part of the light pattern.

In this case, for projecting the first part of the light pattern, the projector may be arranged for example apart from the projector that is used for projecting the second part of the light pattern. As a result, a stereoscopic projection of the first and of the second part of the light pattern onto the examination subject may be facilitated. Furthermore, as a result, accuracy in the mapping of the, for example current, positioning of the examination subject and/or of the surface shape of the examination subject may be improved using a proposed representation apparatus. During an examination and/or treatment on an examination subject, at least one of the projectors in the projection unit may be at least partly covered, as a result of which the projection of the respective part of the light pattern may be obstructed. Through the projectors in the projection unit being arranged apart from each other, projection of at least one part of the light pattern onto the examination subject may be ensured.

Furthermore, the first part of the light pattern may include a different pattern compared with the second part of the light pattern. This may be facilitated for example by the differences in the defined projection geometries, that are specified respectively for the first and the second part of the light pattern. The defined projection geometries for the first and the second part of the light pattern may for example be specified such that interference occurs on the surface of the examination subject. By mapping the resulting light pattern by the camera unit in a proposed representation apparatus, a particularly precise mapping of the positioning of the examination subject may be facilitated.

Alternatively, or additionally, the projection unit may be configured for projecting the first and the second part of the light pattern onto the examination subject at separate times (that is, time multiplexing). For example, the first and the second part of the light pattern may be projectable onto the examination subject alternately with a specified repeat frequency. As a result, a distinctness may be ensured even with at least partially identical light wavelength zones in the first and second part of the light pattern.

The proposed representation apparatus may be configured for, for example separate and/or simultaneous, mapping of the first and second part of the light pattern.

In an embodiment of the proposed projection apparatus, the projector may generate the first part of the light pattern within a first light wavelength zone. Furthermore, the at least one further projector may generate the second part of the light pattern within a second light wavelength zone. The first and the second light wavelength zone may be at least partly different (that is, color multiplexing).

Here, a light wavelength zone may include a single light wavelength and/or a zone, for example a coherent zone of light wavelengths from a light spectrum. For example, a light color may be specifiable by specifying a light wavelength zone for the first and the second part of the light pattern respectively. By projecting the first and second part of the light pattern with an at least partly different light wavelength zone in each case, a simultaneous and separable mapping of the first and the second part may be facilitated using the camera unit in a proposed representation apparatus.

Furthermore, a light mixing may occur on the surface of the examination subject. There is a superimposed projection of the first and second part of the light pattern. By mapping the resulting light pattern, a particularly precise determination of the positioning of the examination subject and/or of the surface shape of the examination subject may ensue using the defined projection geometries of the first and second part of the light pattern.

A third aspect relates to a further representation apparatus for representing an augmented and/or virtual reality. The further representation apparatus includes a sensor unit for mapping a relative spatial positioning of at least one proposed representation apparatus. The further representation apparatus is further configured for adjusting the representation of the augmented and/or virtual reality using the light pattern mapped by the at least one representation apparatus, the defined projection geometry, and the mapped relative spatial positioning of the at least one representation apparatus.

The advantages of the further representation apparatus substantially correspond to the advantages of the representation apparatus. For example, the further representation apparatus may be a proposed representation apparatus. Features, advantages, or alternative embodiments referred to here may also be equally well applied to the other claimed subject matter and vice versa.

The sensor unit in the further representation apparatus is configured for mapping the relative spatial positioning of the at least one proposed representation apparatus. The sensor unit may be a camera, for example a 2D camera and/or an omni-directional camera and/or a 3D-camera, for example a stereo camera and/or a depth camera and/or a time-of-flight camera (TOF camera), and/or an electromagnetic sensor, for example a tracking system, and/or an ultrasound sensor, for example an ultrasound-based tracking system, and/or an optical sensor. The relative spatial positioning mapped by the sensor unit in the at least one representation apparatus may provide information relating to a spatial position of the at least one representation apparatus with regard to the further representation apparatus and/or information relating to an alignment of the at least one representation apparatus, for example of the direction of view, with respect to the further representation apparatus, for example regarding the direction of view of the further representation apparatus. As a result, a coordinate system of the further representation apparatus may be registered with the coordinate system of the at least one representation apparatus.

Furthermore, the sensor unit may, for example additionally, be configured for mapping a relative positioning of the projection unit with respect to the further representation apparatus. As a result, a higher precision may be achieved when adjusting the representation of the augmented and/or virtual reality to the examination subject.

Furthermore, the further representation apparatus may be configured for receiving the light pattern mapped by the at least one representation apparatus. For this purpose, the at least one representation apparatus may provide a signal, for example real time image data, that are provided by the camera unit, to the further representation apparatus. Alternatively, or additionally, the further representation apparatus may receive a transformation instruction for registering the coordinate system of the at least one representation apparatus with the coordinate system of the examination subject and/or the projection apparatus. By using the mapped relative spatial positioning, the defined projection geometry and the light pattern mapped by the at least one representation apparatus, the coordinate system of the further representation apparatus may be registered with the coordinate system of the examination subject, for example indirectly. As a result, the representation of the augmented and/or virtual reality of the further representation apparatus may be precisely adjustable to the examination subject, for example to a positioning of the examination subject.

The proposed further representation apparatus facilitates an indirect registration between the representation of the augmented and/or virtual reality with the examination subject. In this case, it is sufficient if the further representation apparatus maps the relative spatial positioning of at least one proposed representation apparatus and receives the light pattern mapped by the at least one representation apparatus. As a result, a financial and computational outlay involved in the simultaneous generation of a respective representation of the augmented and/or virtual reality for a plurality of users may be reduced. In addition, the further representation apparatus itself does not require any mapping of the projected light pattern on the examination subject. As a result, a particularly flexible arrangement and mobility of a user with the further representation apparatus may be facilitated.

In an embodiment of the proposed further representation apparatus, the further representation apparatus may include a further projection unit, that is configured for projecting a further light pattern onto the at least one representation apparatus. Furthermore, the sensor unit may include a further camera unit that is configured for mapping the further light pattern. The sensor unit may be further configured to determine the relative spatial positioning of the at least one representation apparatus using the mapped further light pattern.

The further projection unit may for example have all the advantages and features that have been described with respect to the proposed projection unit. Features, advantages, or alternative embodiments referred to here may also be equally well applied to the other claimed subject matter and vice versa.

For example, the further projection unit may have at least one projector, for example a light projector and/or a laser projector, that is configured for projecting the further light pattern onto the at least one representation apparatus. In addition, the further camera unit may include a camera, for example a 2D camera and/or an omni-directional camera and/or a 3D camera, for example a stereo camera and/or a depth camera and/or a time-of-flight camera (TOF-camera), that is configured for mapping the further light pattern on the at least one representation apparatus. The further light pattern is projected with a defined projection geometry onto the at least one representation apparatus. The at least one representation apparatus may have a defined form, for example geometry and/or spatial extent. By using the defined form of the at least one representation apparatus, the defined projection geometry of the further light pattern and the further light pattern mapped by the camera unit of the further representation apparatus, the relative spatial positioning of the at least one representation apparatus may be determined. Together with the mapping of the relative spatial positioning of the at least one representation apparatus by the sensor unit, greater precision may be achieved as a result. The determination of the relative spatial positioning by the camera unit of the further representation apparatus is for example robust against electromagnetic sources of interference. The further light pattern may be generated by a further projection unit in a light wavelength zone that is not visible for a human user.

A fourth aspect relates to a medical imaging device including a proposed projection unit. The medical imaging device is configured for mapping medical image data from the examination subject. Furthermore, the medical imaging device is configured for providing the medical image data for generating an augmented and/or virtual reality to a proposed representation apparatus and/or a proposed further representation apparatus.

The medical imaging device may be configured, for example as a medical X-ray device and/or as a magnetic resonance tomography unit (MRT) and/or as a computer tomography (CT) unit and/or as an ultrasound device and/or a positron emission tomography (PET) unit.

Furthermore, the provision of the medical image data may include, for example, storage on a computer-readable storage medium and/or transmission to the representation apparatus and/or to the further representation apparatus and/or to a processing unit. For example, the medical image data may be acquired from the examination subject by the medical imaging device such that the medical image data relating to a coordinate system of the examination subject are at least registered at the time of acquisition.

The advantages of the proposed medical imaging device correspond to the advantages of the proposed representation apparatus, of the proposed further representation apparatus and of the proposed projection apparatus. Features, advantages, or alternative embodiments referred to here may also be equally well applied to the other claimed subject matter and vice versa.

A fifth aspect relates to a system including a proposed representation apparatus and a proposed projection unit. The system includes an interface and a processing unit. The processing unit is configured to control the projection unit. Furthermore, the processing unit is configured to provide the defined projection geometry to the representation apparatus by the interface.

The processing unit may be configured to process information and/or data and/or signals from the representation apparatus and/or from the projection unit and/or from a further component of the proposed medical imaging device. The processing unit may be configured for receiving and/or providing the defined projection geometry of the light pattern. By the interface, the processing unit may facilitate a for example wireless and/or instantaneous, communication with the representation apparatus and the projection unit.

The advantages of the proposed system correspond to the advantages of the proposed representation apparatus, of the proposed further representation apparatus and of the proposed projection apparatus. Features, advantages, or alternative embodiments referred to here may also be equally well applied to the other claimed subject matter and vice versa.

In an embodiment of the proposed system, the system may include a proposed further representation apparatus. The interface may be further configured to receive a signal as a function of the light pattern mapped by the representation apparatus. Furthermore, the processing unit may be configured to provide the defined projection geometry and/or provide the signal to the further representation apparatus by the interface.

An, for example wireless and/or instantaneous, communication between the projection unit, the representation apparatus, the further representation apparatus, and the processing unit may be facilitated. The signal may include information regarding the light pattern mapped by the representation apparatus. Through the signal provided by the processing unit to the further representation apparatus, the representation of the augmented and/or virtual reality in the further representation apparatus may be adjusted to the, for example current, positioning of the examination subject.

A sixth aspect relates to an, for example computer-implemented method for the registration of a proposed representation apparatus. In a first step a1), a light pattern may be projected onto the surface of the examination subject by a proposed projection unit. The light pattern includes a defined projection geometry relative to the projection unit. In a second step b1), the light pattern projected onto the surface of the examination subject is mapped at least in segments by the camera unit. In a third step c1), a transformation instruction is determined between a coordinate system of the representation apparatus and a coordinate system of the examination subject using the light pattern that has been mapped and the defined projection geometry. Subsequently, in a fourth step d1), a representation of the augmented and/or virtual reality is adjusted on the basis of the transformation instruction.

The coordinate system of the representation apparatus may be registered with the coordinate system of the examination subject using the transformation instruction determined in step c1). Subsequently, in this case, the representation of the augmented and/or virtual reality may be adjusted to the for example current positioning of the examination subject, for example of the coordinate system of the examination subject. The representation of the augmented and/or virtual reality may be registered for example with the coordinate system of the examination subject on the basis of the transformation instruction.

The steps b1) to d1) may be carried out repeatedly in the representation of the augmented and/or virtual reality, for example continuously. As a result, a precise and flexible adjustment of the representation of the augmented and/or virtual reality may be facilitated both in the case of an altered spatial positioning of the examination subject and also in the event of an altered spatial positioning of the representation apparatus.

The advantages of the proposed method for the registration of a proposed representation apparatus correspond to the advantages of the proposed representation apparatus, of the proposed further representation apparatus and of the proposed projection apparatus and of the proposed system. Features, advantages, or alternative embodiments referred to here may also be equally well applied to the other claimed subject matter and vice versa.

A seventh aspect relates to a, for example computer-implemented, method for the registration of a proposed further representation apparatus. In a first step a2), a light pattern is projected onto the surface of the examination subject using a proposed projection unit. The light pattern includes a defined projection geometry relative to the projection unit. In a second step b2), the light pattern projected into the surface of the examination subject by the camera unit maps at least one proposed representation apparatus at least in segments. Furthermore, in a third step c2), a relative spatial positioning of the at least one representation apparatus is mapped by the sensor unit of the further representation apparatus. Subsequently, in a fourth step d2), a further transformation instruction is determined between a coordinate system of the further representation apparatus and a coordinate system of the examination subject, using the light pattern mapped by the at least one representation apparatus, the defined projection geometry, and the mapped relative spatial positioning of the at least one representation apparatus. In a fifth step e2), the representation of the augmented and/or virtual reality is adjusted on the basis of the further transformation instruction.

The coordinate system of the further representation apparatus may be registered with the coordinate system of the examination subject using the further transformation instruction determined in step d2). The registration between the coordinate systems may ensue, for example indirectly, via the coordinate system of the at least one representation apparatus. The representation of the augmented and/or virtual reality may be registered for example with the coordinate system of the examination subject on the basis of the further transformation instruction.

The steps b2) to e2) may be carried out repeatedly during the representation of the augmented and/or virtual reality, for example continuously. As a result, a precise and flexible adjustment of the representation of the augmented and/or virtual reality may be facilitated both in the case of an altered spatial positioning of the examination subject and also in the event of an altered spatial positioning of the further representation apparatus.

The advantages of the proposed method for the registration of a proposed further representation apparatus correspond to the advantages of the proposed representation apparatus, of the proposed further representation apparatus and of the proposed projection apparatus, and of the proposed system. Features, advantages, or alternative embodiments referred to here may also be equally well applied to the other claimed subject matter and vice versa.

Furthermore, the proposed system, for example the processing unit, may be configured to carry out a proposed method for the registration of a representation apparatus and/or for the registration of further representation apparatus.

An eighth aspect relates to a computer program product, that includes a program and that may be loaded directly into a memory of a programmable computation unit, and includes programming code, for example, libraries and auxiliary functions in order to carry out a method for registering a representation apparatus and/or a method for registering a further representation apparatus when the computer program product is executed. The computer program product may include software with a source code that still has to be compiled and bound or merely interpreted, or include an executable software code, that only has to be loaded into the processing unit for execution. By the computer program product, the method for the registration of a further representation apparatus may be carried out quickly, in an identically reproducible manner and robustly. The computer program product is configured such that it may carry out the process steps using the processing unit. The processing unit must in each case include the prerequisites for an appropriate main memory, an appropriate graphics card or an appropriate logic unit, such that the respective process steps may be carried out efficiently.

The computer program product is stored for example, on a computer-readable medium, or on a network or server from which it may be loaded into the processor of a processing unit, which processor is directly connected to the processing unit or may be configured as part of the processing unit. Furthermore, control data relating to the computer program product may be stored on an electronically readable data carrier. The control data in the electronically readable data carrier may be configured such that they carry out a method when the data carrier is used in a processing unit. Examples of electronically readable data carriers are a DVD, a magnetic tape, or a USB stick, on which electronically readable control data, for example software, is stored. When this control data is read by the data carrier and stored in a processing unit, all the embodiments of the methods described in the aforementioned may be carried out. Embodiments may also take as its point of departure the computer-readable medium and/or the electronically readable data carrier. The advantages of the proposed computer program product correspond to the advantages of the proposed method.

### BRIEF DESCRIPTION OF THE FIGURES

FIG. 1 depicts a schematic representation of an embodiment of a system including a representation apparatus, a projection unit, and a processing unit.

FIG. 2 depicts a schematic representation of an embodiment of a medical imaging device.

FIG. 3 depicts a schematic representation of an embodiment of a system where a reflection unit is used for indirect mapping of a projected light pattern.

FIG. 4 depicts a schematic representation of an embodiment of a projection unit including two projectors.

FIGS. 5 and 6 depict schematic representations of various embodiments of a further representation apparatus.

FIG. 7 depicts a schematic representation of an embodiment of a method for the registration of a representation apparatus.

FIG. 8 depicts a schematic representation of an embodiment of a method for the registration of a further representation apparatus.

### DETAILED DESCRIPTION

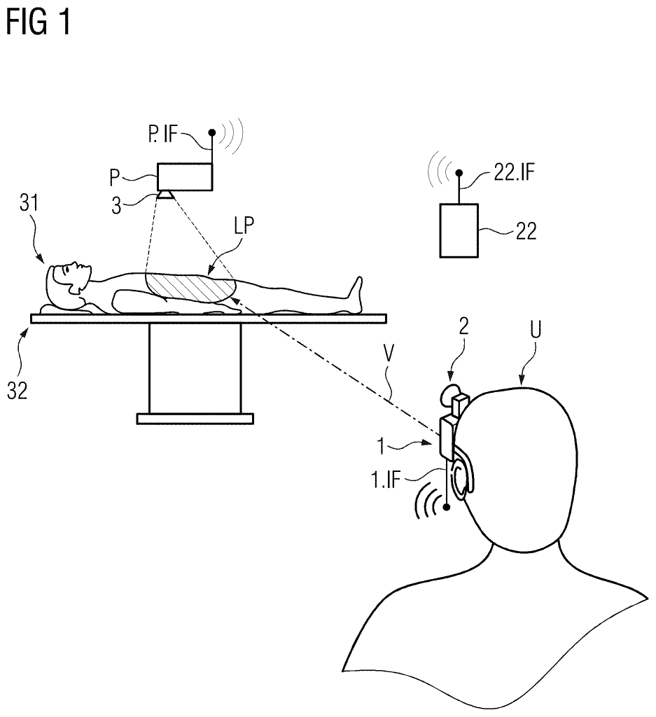

FIG. 1 depicts in schematic form an embodiment of a proposed system including a representation apparatus, a projection unit, and a processing unit. The representation apparatus 1 may include for example a portable display unit, that may for example be carried by a user, which unit is configured to display the augmented and/or virtual reality (abbreviated to AR and VR respectively). The representation apparatus 1, for example the display unit, may be at least partly configured to be transparent. The representation apparatus 1 may be configured such that it may be carried by a user U at least partly within a field of view of the user U. For this purpose, the representation apparatus 1 may be configured as pair of glasses, for example as data glasses. Furthermore, the representation apparatus 1 may include a camera unit 2, that is configured for mapping, at least in segments, an examination subject 31 arranged in a direction of view V of the representation apparatus 1. The camera unit 2 may be at least partly incorporated in the representation apparatus 1, for example in the data glasses. The examination subject 1 may be arranged on a patient positioning apparatus 32. Furthermore, the camera unit 2 may be configured for mapping a light pattern LP that is projected onto the examination subject 31 using the projection unit 2. The light pattern LP may include a defined projection geometry relative to the projection unit P. Furthermore, the representation apparatus 1 may be configured to adjust the representation of the augmented and/or virtual reality using the mapped light pattern LP and the defined projection geometry.

The projection unit P for projecting the light pattern LP onto the surface of the examination subject 31 may include a projector 3. The projector 3 may be configured for projecting the light pattern LP onto the surface of the examination subject 31. Moreover, the projection unit P may be configured to provide, for example wirelessly, the defined projection geometry to the representation apparatus 1 and/or to a processing unit 22. For this purpose, the projection unit P may include an interface P.IF. Furthermore, the representation apparatus 1 may include an interface 1.IF. The interface LIF may be configured for receiving a signal as a function of the defined projection geometry. In a similar way, the processing unit 22 may include an interface 22.IF, that is configured for sending and/or receiving signals to the representation apparatus 1 and/or the projection unit P.

Moreover, the processing unit 22 may be configured for controlling the projection unit P. For this purpose, the processing unit may send an appropriate signal to the projection unit P.

Furthermore, the representation apparatus 1 may be configured for reconstructing a 3D model of a surface of the examination subject 31 using the mapped light pattern LP and the defined projection geometry. The representation of the augmented and/or virtual reality may be additionally adjustable using the 3D model.

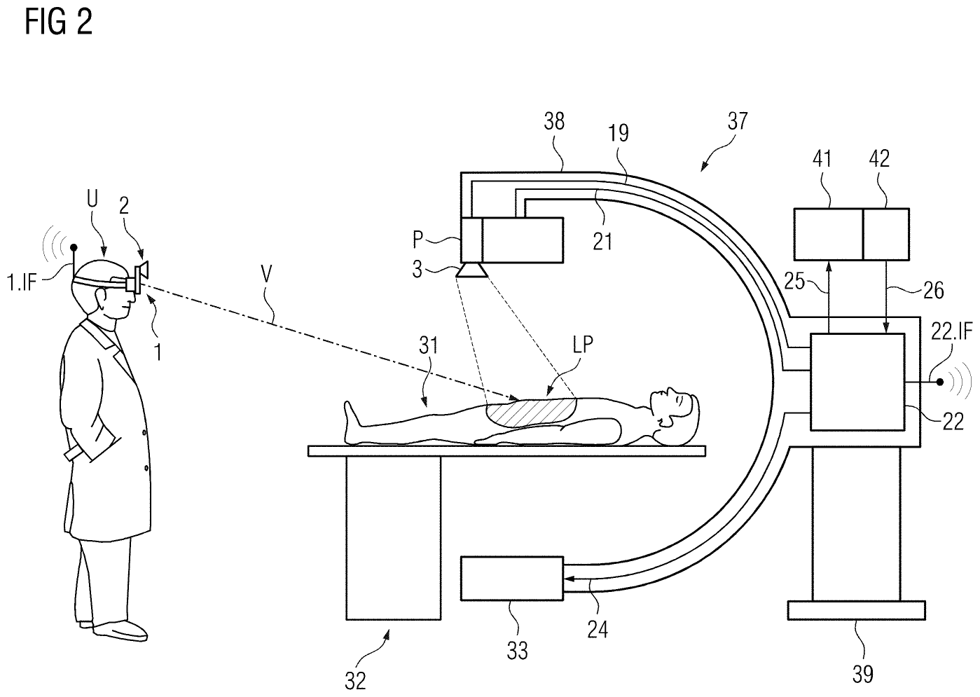

FIG. 2 depicts a schematic representation of an embodiment of a proposed medical imaging device. The medical imaging device may be configured, for example, as a medical C-arm X-ray device 37. Furthermore, the medical C-arm X-ray device 37 may include the processing unit 22. The medical imaging device 37, for example the processing unit 22, is configured to carry out the proposed method for the registration of a representation apparatus 1 and/or for the registration of a further representation apparatus.

The medical C-arm X-ray device 37 additionally includes a detector unit 34 and an X-ray source 33. To record medical image data, the arm 38 of the medical C-arm X-ray device 37 may be moveably mounted around one or a plurality of axes.

Furthermore, the medical C-arm X-ray device 37 may include a movement device 39, that facilitates a movement of the C-arm X-ray device 37 in the space. In addition, the projection unit P may be arranged on the medical C-arm X-ray device 37 and/or be at least partly incorporated in the C-arm X-ray device 37. As a result, an inherent registration between a coordinate system of the projection unit P and a coordinate system of the medical C-arm X-ray device 37, for example of the medical image data, may be facilitated.

To record the medical image data from the examination subject 31, the processing unit 22 may send a signal 24 to the X-ray source 33. Then the X-ray source 33 may emit an X-ray beam, for example a cone beam and/or a fan beam and/or a parallel beam. When, after an interaction with an examination region of the examination subject 31 that is to be mapped, the X-ray beam impinges on a surface of the detector unit 34, the detector unit 34 may transmit a signal 21 to the processing unit 22. The processing unit 22 may receive the medical image data, for example using the signal 21. Furthermore, the processing unit 22 may provide the medical image data to generate the augmented and/or virtual reality to the representation apparatus 1. For this purpose, the processing unit 22 may send an appropriate signal, for example wirelessly, to the interface 1.IF of the representation apparatus 1. To control the projection unit P, the processing unit 22 may send a signal 19 to the projection unit P. The projection unit, for example the projector 3, may project the light pattern LP with the defined projection geometry onto the examination subject 31 as a function of the signal 19 from the processing unit 22.

Furthermore, the medical C-arm X-ray device 37 may include an input unit 42, for example a keyboard, and/or a display unit 41, for example a monitor and/or display. The input unit 42 may preferably be incorporated in the display unit 41, for example in a capacitive input display. Here, through an input by an operator on the input unit 42, a control of the medical C-arm X-ray device 37 may be facilitated. For this purpose, the input unit 42 may, for example, transmit a signal 26 to the processing unit 22.

Furthermore, the display unit 41 may be configured to display information and/or graphic representations of information from the medical C-arm X-ray device 37 and/or from the processing unit 22 and/or from further components. For this purpose, the processing unit 22 may, for example, send a signal 25 to the display unit 41.

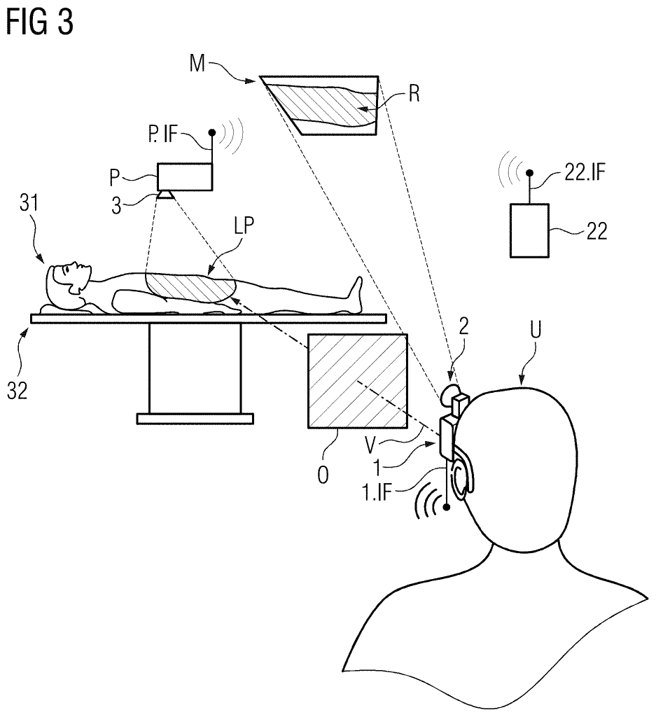

In the embodiment shown schematically in FIG. 3, the camera unit 2 may be configured for the indirect mapping of the light pattern LP via a reflection unit M. The camera unit 2 may map an image R of the light pattern LP on the reflection unit M. The reflection unit M may have a known spatial positioning relative to the projection unit P. The reflection unit M may be configured, for example, as a mirror. Furthermore, the reflection unit M may be arranged apart from the examination subject 31 such that the reflection unit M is substantially arranged outside a direct direction of view V of the representation apparatus 1 towards the examination subject 31. When there is a visual obstruction, for example due to a physical object O, in the direction of view V of the representation apparatus 1, the representation of the augmented and/or virtual reality may continue to be ensured via the indirect mapping of the light pattern LP.

Alternatively, or additionally, the camera unit 2 may be configured for mapping the positioning of the reflection unit M. The positioning of the reflection unit M may be arranged using a defined shape and size of the reflection unit M and/or may be mapped by at least one marker object, that is arranged on and/or incorporated in the reflection unit M, by the camera unit 2. For example, there is a substantially rectangular and/or circular defined shape and known size of the reflection unit M, for example by distorting and/or tilting the perspective, the positioning of the reflection unit M may be determinable by an at least two-dimensional mapping using the camera unit 2.

Furthermore, the reflection unit M may be arranged to be moveably defined, the positioning of the reflection unit M for example being able to be adjustable by tilting and/or rotating and/or by a translatory movement. The adjustment of the positioning of the reflection unit M may be facilitated by a motor unit, for example by an electric motor and/or a pneumatic motor. The processing unit 22 may send an appropriate signal to the reflection unit M. The positioning of the reflection unit may be adjustable as a function of the positioning of the representation apparatus 1. As a result, it may be ensured that the light pattern LP is constantly mappable by the camera unit 2 indirectly via the reflection unit M.

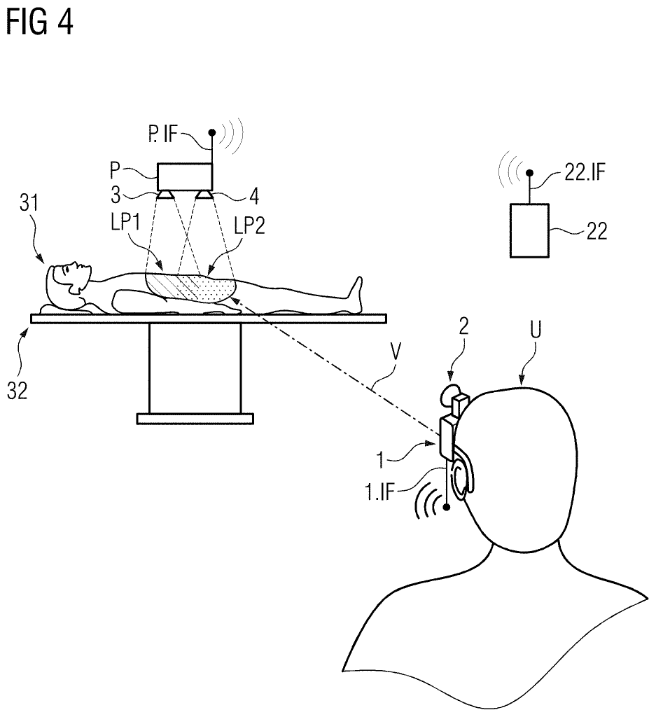

FIG. 4 depicts a further embodiment of the proposed projection unit P. The light pattern LP includes a first part LP1 and a second part LP2. The projector 3 may be configured for projecting the first part of the light pattern LP1 onto the surface of the examination subject. Furthermore, the projection unit P may include at least one further projector 4, that is configured for projecting the second part of the light pattern LP2 onto the surface of the examination subject. The second part of the light pattern LP2 may have a different defined projection geometry from the first part of the light pattern. In addition, the projector 3 may generate the first part of the light pattern LP1 within a first light wavelength zone. Furthermore, the at least one further projector 4 may generate the second part of the light pattern LP2 within a second light wavelength zone. The first and the second light wavelength zone may be at least partly different.

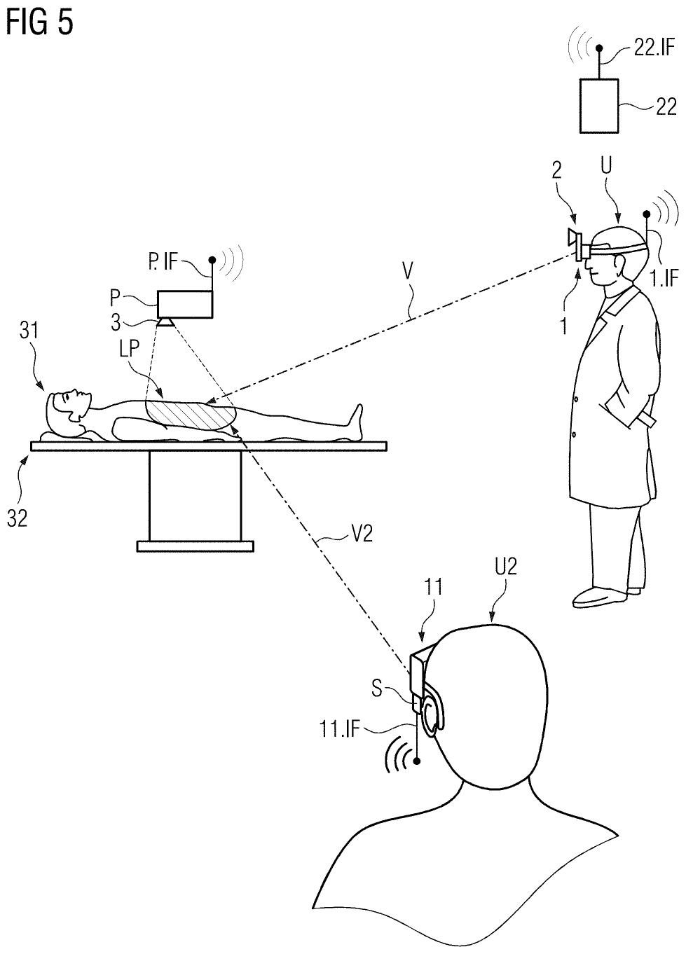

FIG. 5 depicts a further representation apparatus 11 for representing an augmented and/or virtual reality in schematic form. The further representation apparatus 11 may include, for example by analogy with the at least one representation apparatus 1, a portable display unit, for example capable of being carried by a further user U2, which unit is configured to display the augmented and/or virtual reality. The further representation apparatus 11 may be configured such that it is able to be carried by the further user U2 at least partly within a field of view of the further user U2. For this purpose, the further representation apparatus 11 may be configured as a pair of glasses, for example as data glasses.

The further representation apparatus 11 may include a sensor unit S for mapping a relative spatial positioning of at least one representation apparatus 1. Furthermore, the further representation apparatus 11 may be configured for adjusting the representation of the augmented and/or virtual reality using the light pattern mapped by the at least one representation apparatus 1, the defined projection geometry and the mapped relative spatial positioning of the at least one representation apparatus 1. Furthermore, the further representation apparatus 11 may include an interface 11.IF, that is configured, for receiving, for example wirelessly, the defined projection geometry and/or the light pattern LP mapped by the at least one representation apparatus 1. For this purpose, the at least one representation apparatus 1 and/or the processing unit 22 may transmit a corresponding signal to the further representation apparatus 11.

As a result, an indirect registration between a coordinate system of the further representation apparatus 11 and a coordinate system of the examination subject 31 may be facilitated.

In the embodiment shown schematically in FIG. 6, the further representation apparatus 11 may include a further projection unit P2, that is configured for projecting a further light pattern LPX onto the at least one representation apparatus 1 and/or onto the projection unit P. Furthermore, the sensor unit S may include a further camera unit K2, that is configured for mapping the further light pattern LPX. The sensor unit S may additionally be configured to determine the relative spatial positioning of the at least one representation apparatus 1 and/or of the projection unit P, using the mapped further light pattern LPX. The further camera unit K2 may include at least one camera, for example a 2D camera and/or an omni-directional camera and/or a 3D-camera, for example a stereo camera and/or a depth camera and/or a time-of-flight camera, that is configured for mapping the further light pattern LPX. As a result, a precise and reliable mapping of the relative positioning of the at least one representation apparatus 1 may be facilitated. Furthermore, the adjustment of the representation of the augmented and/or virtual reality of the further representation apparatus 11 may be ensured even in the event of a visual obstruction within the direction of view V2 of the further representation apparatus 11.

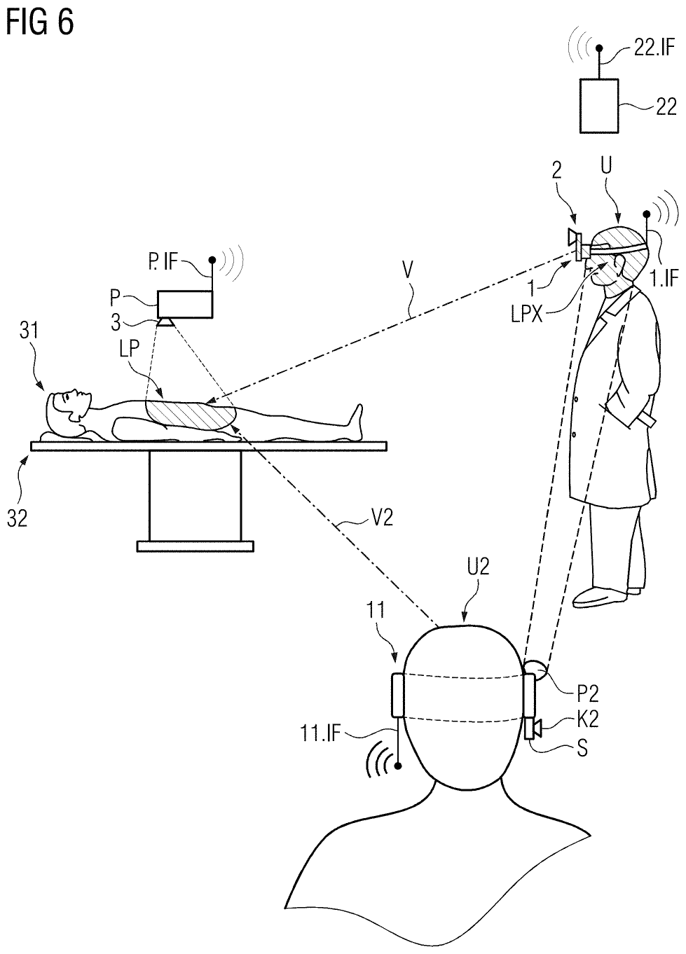

FIG. 7 depicts a schematic representation of an embodiment of the proposed method for the registration of a representation apparatus 1 with an examination subject 31. In a first step, a1) the light pattern LP may be projected PROJ-LP onto the surface of the examination subject 31 by the projection unit P. Furthermore, in the second step b1), at least one segment of the light pattern LP that is projected onto the surface of the examination subject 31 may be mapped CAP-LP by the camera unit 2. In a third step c1), a transformation instruction may be determined DET-T between the coordinate system of the representation apparatus 1 and the coordinate system of the examination subject 31, using the mapped light pattern LP and the defined projection geometry. Subsequently, the representation of the augmented and/or virtual reality may be adjusted ALT-VISU in a fourth step d1) on the basis of the transformation instruction.

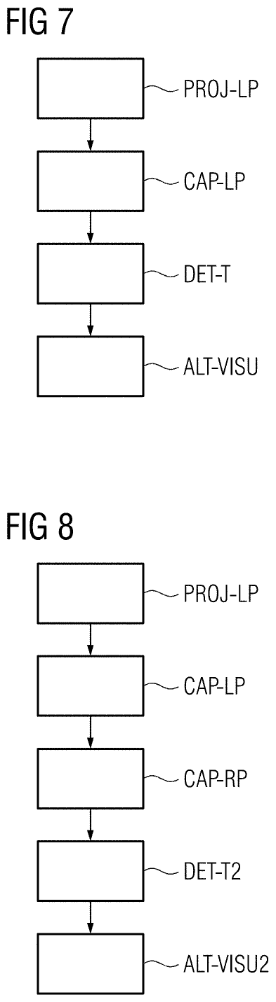

FIG. 8 depicts in schematic form an embodiment of the proposed method for registering a further representation apparatus 11 with an examination subject 31. In a first step a2), the light pattern LP may be projected PROJ-LP onto the surface of the examination subject 31 by the projection unit P. Furthermore, in a second step b2), at least one segment of the light pattern LP that is projected onto the surface of the examination subject 31 may be mapped CAP-LP by the camera unit 2 of the at least one representation apparatus 1. Subsequently, in a third step c2), a relative spatial positioning of the at least one representation apparatus 1 may be mapped CAP-RP by the sensor unit S of the further representation apparatus 11. Furthermore, in a fourth step d2), a further transformation instruction between the coordinate system of the further representation apparatus 11 and the coordinate system of the examination subject 31 may be determined DET-T2 using the light pattern LP mapped by the at least one representation apparatus 1, the defined projection geometry and the mapped relative spatial positioning of the at least one representation apparatus 1. Subsequently, in a fifth step e2), the representation of the augmented and/or virtual reality of the further representation apparatus 11 may be adjusted ALT-VISU2 on the basis of the further transformation instruction.

The schematic representations in the figures described do not in any way represent a scale or size ratio.

Finally, it is once again pointed out that the detailed methods described in the aforementioned and the apparatus that is shown involve merely exemplary embodiments that may be modified in a variety of ways by a person skilled in the art without departing from the scope of the invention. Furthermore, the use of the indefinite article “a” or “an” does not preclude the relevant features from being present in plurality. Likewise, the terms “unit” and “element” do not preclude the relevant components from consisting of a plurality of interacting components that may optionally also be spatially distributed.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A system comprising a first representation apparatus for representing a first augmented reality, a first virtual reality, or the first augmented reality and the first virtual reality, the first representation apparatus comprising:
a camera unit configured for mapping, at least in segments, an examination subject arranged in a direction of view of the first representation apparatus;
wherein the camera unit is further configured for mapping a light pattern that is projected onto the examination subject by a first projection unit;
wherein the light pattern includes a defined projection geometry relative to the first projection unit;
wherein the first representation apparatus is configured for adjusting a representation of the first augmented reality or the first virtual reality using the mapped light pattern and the defined projection geometry.

2. The system of claim 1, wherein the first representation apparatus is further configured for reconstructing a three-dimensional model of a surface of the examination subject using the light pattern and the defined projection geometry;
wherein the representation of the first augmented reality or the first virtual reality is further adjustable by the three-dimensional model.

3. The system of claim 1, wherein the first representation apparatus is configured for graphically representing, as the first augmented reality or the first virtual reality, medical image data from the examination subject that is acquired or provided by a medical imaging device.

4. The system of claim 1, wherein the camera unit is further configured for mapping the light pattern indirectly via a reflection unit; wherein the reflection unit includes a known spatial positioning relative to the first projection unit.

5. The system of claim 1, further comprising:
a second representation apparatus configured to represent a second augmented reality, a second virtual reality, or the second augmented reality and the second virtual reality, the second representation apparatus comprising a sensor unit configured to map a relative spatial positioning of the first representation apparatus;
wherein the second representation apparatus is configured to adjust the representation of the second augmented reality, the second virtual reality, or the second augmented reality and the second virtual reality using the light pattern, the defined projection geometry, and the mapped relative spatial positioning of the first representation apparatus.

6. The system of claim 5, wherein the second representation apparatus comprises a second projection unit that is configured to project a further light pattern onto the first representation apparatus;
wherein the sensor unit comprises a further camera unit that is configured to map the further light pattern,
wherein the sensor unit is configured to determine the relative spatial positioning of the first representation apparatus using the mapped further light pattern.

7. The system of claim 1, wherein the first projection unit comprises:
a projector configured for projecting the light pattern onto a surface of the examination subject; wherein the light pattern includes a defined projection geometry relative to the first projection unit; wherein the first projection unit is configured to provide the defined projection geometry to the first representation apparatus configured for representing the first augmented reality, the first virtual reality, or the first augmented reality and the first virtual reality.

8. The system of claim 7, wherein the light pattern comprises a first part and a second part, wherein the projector is configured for projecting the first part of the light pattern onto the surface of the examination subject;
wherein the first projection unit comprises at least one further projector that is configured for projecting the second part of the light pattern onto the surface of the examination subject;
wherein the second part of the light pattern includes a different defined projection geometry from the first part of the light pattern.

9. The system of claim 8, wherein the projector is configured to generate the first part of the light pattern within a first light wavelength zone; wherein the at least one further projector is configured to generate the second part of the light pattern within a second light wavelength zone; wherein the first and the second light wavelength zone are at least partly different.

10. A medical imaging device configured to provide medical image data for generating an augmented reality or virtual reality, the medical imaging device comprising:
a projector configured for projecting a light pattern onto a surface of an examination subject; wherein the light pattern includes a defined projection geometry relative to the projector; wherein the projector is configured to provide the defined projection geometry to a first representation apparatus; and
the first representation apparatus configured for representing a first augmented reality, a first virtual reality, or the first augmented reality and the first virtual reality, the first representation apparatus including a camera unit configured for mapping, at least in segments, the examination subject arranged in a direction of view of the first representation apparatus, wherein the camera unit is further configured for mapping the light pattern that is projected onto the examination subject by the projector, wherein the first representation apparatus is configured for adjusting a representation of the first augmented reality, the first virtual reality, or the first augmented reality and the first virtual reality using the mapped light pattern and the defined projection geometry.

11. The medical imaging device of claim 10, wherein the first representation apparatus is further configured for reconstructing a three-dimensional model of a surface of the examination subject using the light pattern and the defined projection geometry;
wherein the representation of the first augmented reality, the first virtual reality, or the first augmented reality and the first virtual reality is further adjustable by the three-dimensional model.

12. The medical imaging device of claim 10, wherein the first representation apparatus is configured for graphically representing, as the first augmented reality, the first virtual reality, or the first augmented reality and the first virtual reality, medical image data from the examination subject that is acquired or provided by the medical imaging device.

13. The medical imaging device of claim 10, wherein the camera unit is further configured for mapping the light pattern indirectly via a reflection unit; wherein the reflection unit includes a known spatial positioning relative to the projector.

14. The medical imaging device of claim 10, further comprising:
a second representation apparatus comprising:
a sensor unit configured to map a relative spatial positioning of the first representation apparatus;
wherein the second representation apparatus is configured to adjust a representation of a second augmented reality, a second virtual reality, or the second augmented reality and the second virtual reality using the light pattern, the defined projection geometry, and the mapped relative spatial positioning of the first representation apparatus.

15. The medical imaging device of claim 14, wherein the second representation apparatus comprises a further projection unit that is configured to project a further light pattern onto the first representation apparatus;
wherein the sensor unit comprises a further camera unit that is configured to map the further light pattern,
wherein the sensor unit is configured to determine the relative spatial positioning of the first representation apparatus using the mapped further light pattern.

16. A method for registering a representation apparatus configured to provide an augmented reality or virtual reality with an examination subject, the method comprising:
projecting a light pattern onto a surface of the examination subject using a projection unit, wherein the light pattern includes a defined projection geometry relative to the projection unit;
mapping, at least in segments, of the light pattern that is projected onto the surface of the examination subject by a camera unit;
determining a transformation instruction between a coordinate system of the representation apparatus and a coordinate system of the examination subject using the mapped light pattern and the defined projection geometry; and
adjusting a representation of the augmented reality or the virtual reality as a function of the transformation instruction.

17. The method of claim 16, further comprising:
mapping of a relative spatial positioning of the representation apparatus using a sensor unit in a further representation apparatus;
determining a further transformation instruction between a coordinate system of the further representation apparatus and a coordinate system of the examination subject using the mapped light pattern and the defined projection geometry, and the mapped relative spatial positioning of the representation apparatus; and
adjusting a further augmented reality, a further virtual reality, or the further augmented reality and further virtual reality represented by the further representation apparatus using the mapped light pattern and the defined projection geometry based on the further transformation instruction.
